(19) United States
(12) Patent Application Publication
(10) Pub. No.: US 2008/0090251 A1
(43) Pub. Date: Apr. 17, 2008

(54) Assays For Superantigens

(75) Inventors: Bent Karsten Jakobsen, Oxfordshire (GB); Nicholas Jonathan Pumphrey, Oxfordshire (GB)

Correspondence Address: BANNER & WITCOFF, LTD., 1100 13th STREET, N.W., SUITE 1200, WASHINGTON, DC, 20005-4051, US

(73) Assignee: MEDIGENE LIMITED, ABINGDON (GB)

(21) Appl. No.: 11/792,538

(22) PCT Filed: Oct. 31, 2005
(86) PCT No.: PCT/GB05/04197

§ 371 Date: Nov. 14, 2007

(30) Foreign Application Priority Data
Dec. 16, 2004 (GB) 0427585.5

(51) Int. Cl. G01N 33/53; C07K 14/74

(52) U.S. Cl. 435/7.1; 530/350; 435/7.92

(57) ABSTRACT

The present invention provides a superantigen quality control assay, particularly for SEA-E120, comprising incubating a standard amount of a superantigen-containing test sample with a standard amount of a soluble TCR which binds the superantigen, separating unbound TCR from the resultant superantigen/TCR-containing sample, quantifying the TCR bound in that sample, and comparing that result with a reference result characterising a control superantigen-containing sample. Also provided are soluble TCRs useful as reagents in said assay.

### BACKGROUND TO THE INVENTION

The present invention relates to a superantigen assay, particularly for SEA-E120, comprising incubating a standard amount of a superantigen-containing test sample with a standard amount of a soluble T cell receptor (TCR) which binds the superantigen, separating unbound TCR from the resultant superantigen/TCR-containing sample, quantifying the TCR bound in that sample, and comparing that result with a reference result characterising a control superantigen-containing sample. Soluble TCR(s) useful as reagents in said assay also form part of the invention.

Superantigens and superantigen-containing compositions are currently being investigated as therapeutic agents. Such therapeutics will require quality control testing as part of the manufacturing process thereof. The assay and reagents disclosed herein will be use of in meeting this need.

The superantigen assays disclosed herein can be carried out using a number of different assay formats. These formats include, but are not limited to; enzyme-linked immunosorbent assays (ELISAs) or interfacial optical assays. These methods rely on the use of soluble T cell receptors (TCRs) and provide novel means of assessing superantigen-containing samples.

### BRIEF DESCRIPTION OF THE INVENTION

This invention makes available for the first time a superantigen assay comprising incubating a standard amount of a superantigen-containing test sample with a standard amount of a soluble TCR which binds the superantigen, separating unbound TCR from the resultant superantigen/TCR-containing sample, quantifying the TCR bound in that sample, and comparing that result with a reference result characterising a control superantigen-containing sample. Soluble TCR(s) useful as reagents in said assay are also made available. Such methods and reagents will be of value as quality control measures during the production of these compositions.

### DETAILED DESCRIPTION OF THE INVENTION

The present invention provides a superantigen assay comprising incubating a standard amount of a superantigen-containing test sample with a standard amount of a soluble TCR which binds the superantigen, separating unbound TCR from the resultant superantigen/TCR-containing sample, quantifying the TCR bound in that sample, and comparing that result with a reference result characterising a control superantigen-containing sample. Soluble TCR(s) useful as reagents in said assay are also made available. Such assays and reagents will be of value as quality control measures during the production of superantigens and superantigen-containing compositions. For example, the modified superantigen SEA-E120 (SEQ ID NO: 21).

One aspect of the invention is provided by a heterodimeric TCR (dTCR) or single-chain TCR (scTCR) comprising SEQ ID NO: 29 which binds to SEA- E120 having SEQ ID NO: 21. SEQ ID NO: 29 is a TCR β chain variable region. The variable region being that part of a TCR β chain not encoded by one of the two functional TCR β chain constant genes. (i.e. TRBC1 or TRBC2)

Another aspect of the invention is provided by a dTCR or scTCR comprising the TCR β chain sequence of SEQ ID NO: 2 which binds to SEA- E120 having SEQ ID NO: 21.

A further aspect of the invention is provided by a dTCR comprising the TCR α chain amino acid sequence of SEQ ID NO: 1 and the TCR β chain sequence of SEQ ID NO: 2.

Superantigens are bacterial or viral proteins which cause immuno-stimulation by cross-linking Class II MHC molecules on the surface of antigen presenting cells (APCs) to TCRs of a defined subset of β chain variable domains. This cross-linking causes polyclonal T cell activation leading to a massive release of cytokines such as IL-2 and TNF-β which can cause lethal toxic shock syndrome. (Li et al., (1999) Annu Rev Immunol 17 435-466) and (Baker et al., (2004) Int J Med Microbiol. 293 (7-8) 529-37) provide reviews of the structure and function of superantigens.

The term “superantigen-containing test sample” as used herein is understood to encompass any test sample which contains a superantigen. The superantigen in the test sample may be provided in a purified or isolated form, for example in a form substantially free of other proteins or compounds. Alternatively, the superantigen may be provided in a form wherein the superantigen is associated, covalently or non-covalently, with one or more other protein(s) and/or compound(s).

Superantigen fusion proteins are examples of therapeutically relevant superantigen-containing compositions. These fusions proteins generally comprise a targeting moiety such as an antibody fragment linked to the superantigen. The targeting moiety functions to bind the fusion protein to a disease-associated cell. The superantigen part of the fusion protein then causes binding of T cells to said disease-associated cell thereby inducing an immune response. The following publications provide detailed information relating to a range of superantigen fusion proteins:

U.S. Pat. No. 6,197,299, U.S. Pat. No. 6,692,746, U.S. Pat. No. 6,514,498.EP0998305, WO03094846, (Ueno et al., (2002) Anticancer Res.22 (2A) 769-76), (Takemura et al., (2002) Cancer Immunol Immunother. 51 (1) :33-44) and (Nielsen et al, (2000) J Immunother 23 (1): 146-53)

The quality control assay of the invention provides information that may be used to evaluate whether or not a test sample generally matches a defined quality standard, and/or to assess the extent to which the test sample deviates from a defined quality standard. The results of such assays are typically used to assess test samples as part of a quality assurance programme.

The assay of the invention involves incubating a standard amount of a superantigen-containing test sample with a standard amount of a soluble TCR. Hence each individual assay is carried out using a mixture of a defined amount of the superantigen-containing test sample and soluble TCR. The use of known weight/volume (w/v) concentrations of superantigen-containing test sample and soluble TCR in the preparation and carrying out of each individual assay is one manner by which to ensure these criteria are be met.

The reference result characterising the control superantigen-containing sample is “benchmark” data against which results generated by the assay for the test samples can be compared.

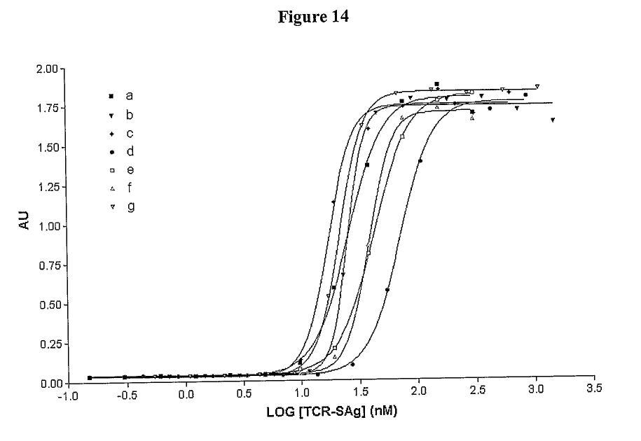

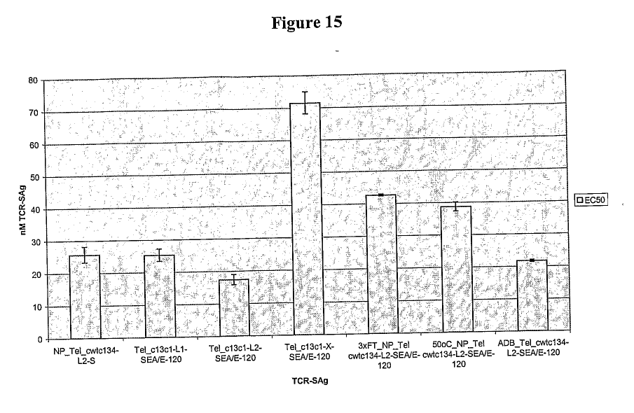

The assay may be performed on a series of aliquots of the superantigen-containing test sample, each aliquot containing a different amount of the said sample, and the bound TCR result for comparison with the reference result is estimated as a function of the individual quantifications of the bound TCR in each aliquot. In one embodiment of this aspect the bound TCR result from each aliquot of a given test sample is used to calculate the concentration of test sample required to cause half-maximal TCR binding (EC50). The EC50 value for a given test sample may be determined by plotting the assay response which is proportional to the bound TCR value obtained for each aliquot against the amount of test sample present in each aliquot. FIG. 14 herein provides a specific example of such a plot for a number of test samples. FIG. 15 herein provides a graphical comparison of the EC50 values of the test samples as determining from the data of FIG. 14.

In one aspect of the invention, the reference result is the result of the same assay performed on a control superantigen-containing sample. This allows the calculation of a relative value for each test sample derived from a comparison of the assay result for said test sample and the control superantigen-containing sample.

The use of a multimeric TCR in these assays forms one aspect of the invention. As is known to those skilled in the art there are a number of means by which multimeric TCR complexes can be formed. These include, but are not limited to, the use of linkers comprising biotin/streptavidin or polyalkylene glycols such as polyethylene glycol. Details of the formation of such multimeric TCR complexes can be found in WO 99/60119 and WO 2004/050705 respectively.

In one aspect of the invention the quantification of the TCR bound in the sample is by an interfacial optical assay (IOA). As will be known to those skilled in the art there are a number of IOA formats which will be suitable for use in the present invention. These include surface plasmon resonance (SPR), total internal reflectance fluorescence (TIRF), resonant mirror (RM) and optical grating coupler sensor (GCS). (Woodbury et al., (1999) J. Chromatog. B. 725 113-137) provides a review of these assay formats. Of course, the reference result need not be acquired at the same time as the test sample result.

In a specific embodiment of this aspect the quantification is by SPR.

SPR-based assays involve immobilising one binding partner (normally the receptor) on a ‘chip’ (the sensor surface) and flowing the other binding partner (normally the ligand), over the chip. The binding of the ligand results in an increase in concentration of protein near to the chip surface which causes a change in the refractive index in that region. The surface of the chip is comprised such that the change in refractive index may be detected by surface plasmon resonance, an optical phenomenon whereby light at a certain angle of incidence on a thin metal film produces a reflected beam of reduced intensity due to the resonant excitation of waves of oscillating surface charge density (surface plasmons). The resonance is very sensitive to changes in the refractive index on the far side of the metal film, and it is this signal which is used to detect binding between the immobilised and soluble proteins. Systems which allow convenient use of SPR detection of molecular interactions, and data analysis, are commercially available. Examples include the Iasys machines (Fisons) and the Biacore machines. The Biacore 3000 system, for example, utilises a sensor chip consisting of four flow cells, thereby allowing the binding of a given soluble ligand to up to four different immobilised proteins in one run.

In one aspect of the invention the quantification of the TCR multimer bound in the sample is by an Enzyme-Linked Immuno-sorption Assay (ELISA). ELISA assays are typically based on the ability of antibodies to specifically bind to their cognate hapten (ligand). The assays utilised an antibody linked with either a detectable signal, such as a fluorophore, or an enzyme, such as horseradish peroxidase (HRP). The fluorophores produce a signal directly in the presence of light of the correct wavelength. Enzymes such as HRP produce a colour change in the presence of its substrate. The strength of these signals is proportion to the amount of the analyte in the sample. ELISAs are often run as two-enzyme “sandwich” assays in which a primary antibody is used to bind to the analyte, followed by a secondary labelled antibody coupled to the desired signalling moiety which then binds to the primary antibody. These systems are popular as there allow the use of secondary labelled antibodies which bind to a wide range primary antibodies, based on the species from which the primary antibody was derived. There are many books which provide details of ELISA, and similar assays, including (Kemeny (1990) A Practical Guide to ELISA, published by Elsevier Science) and (Kemeny et al., (1988) ELISA and Other Solid Phase Immunoassays, published by John Wiley and Sons Ltd).

The preferred ELISA-based methods described herein rely on the use of soluble TCRs to replace antibodies as the ligand binding molecules. This has the advantage of using the superantigens physiological binding partner in the assay.

Soluble TCRs

A number of constructs have been devised for the production of soluble TCRs which will be suitable for use in the assays of the present invention. These constructs fall into two broad classes, single-chain TCRs (scTCRs) and dimeric TCRs (dTCRs). Examples of suitable scTCR constructs include, but are not limited to, those described in WO 2004/033685. Examples of suitable dTCR constructs include, but are not limited to, those described in WO 03/020763, WO099/60120 and WO 2004/048410.

In a further aspect of the invention the TCR for use in the present invention comprises a first polypeptide wherein a sequence corresponding to a TCR α chain variable region sequence fused to the N terminus of a sequence corresponding to a TCR α chain constant region extracellular sequence, and a second polypeptide wherein a sequence corresponding to a TCR β chain variable region sequence fused to the N terminus a sequence corresponding to a TCR β chain constant region extracellular sequence, the first and second polypeptides being linked by a disulfide bond between cysteine residues substituted for Thr 48 of exon 1 of TRAC*01 and Ser 57 of exon 1 of TRBC1*01 or TRBC2*01 or the non-human equivalent thereof.

In an alternative aspect of the invention the dTCR comprises; a TCR α chain comprising a variable α domain, a constant α domain and a first dimerisation motif attached to the C-terminus of the constant α domain, and a TCR β chain comprising a variable β domain, a constant β domain and a first dimerisation motif attached to the C-terminus of the constant β0 domain, wherein the first and second dimerisation motifs easily interact to form a covalent bond between an amino acid in the first dimerisation motif and an amino acid in the second dimerisation motif linking the TCR α chain and TCR β chain together.

As will be obvious to those skilled in the art TCRs of the invention may be provided in forms which further comprise tags, linkers and/or detectable labels. For example, a biotin tag may be added in order to facilitate production of TCR multimers. Example 4 herein details the biotinylation and subsequent tetramerisation of TCRs.

Preferred features of each aspect of the invention are as for each of the other aspects mutatis mutandis. The prior art documents mentioned herein are incorporated to the fullest extent permitted by law.

### EXAMPLES

The invention is further described in the following examples, which do not limit the scope of the invention in any way.

Reference is made in the following to the accompanying drawings in which:

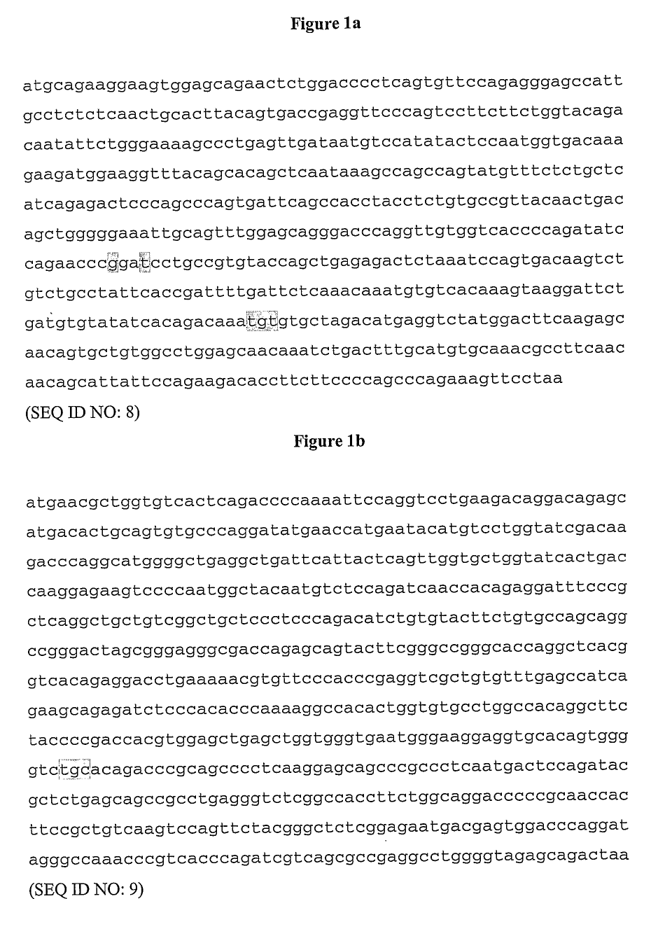

FIGS. 1a and 1b show respectively the nucleic acid sequences of the α and β chains of a soluble A6 TCR, mutated so as to introduce a cysteine codon. The shading indicates the introduced cysteine codons and an introduced BamHl restriction site in the α chain nucleic acid;

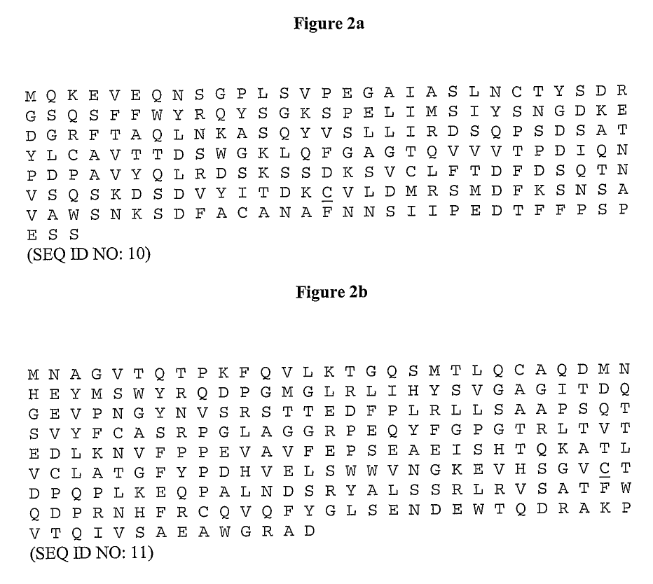

FIG. 2a shows the A6 TCR α chain extracellular amino acid sequence, including the T₄₈→C mutation (underlined) used to produce the novel disulfide inter-chain bond, and FIG. 2b shows the A6 TCR β chain extracellular amino acid sequence, including the S₅₇→C mutation (underlined) used to produce the novel disulfide inter-chain bond;

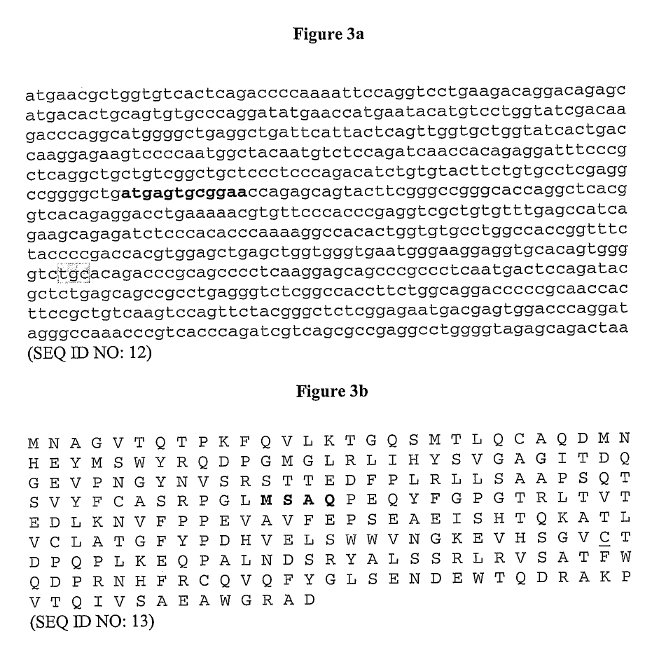

FIGS. 3a and 3b show the DNA and amino acid sequences of the high affinity c134 variant of the A6 Tax TCR β chain mutated to include additional cysteine residues to form a non-native disulfide bond, the introduced cysteine codon is indicated by shading and the affinity increasing mutations are in bold;

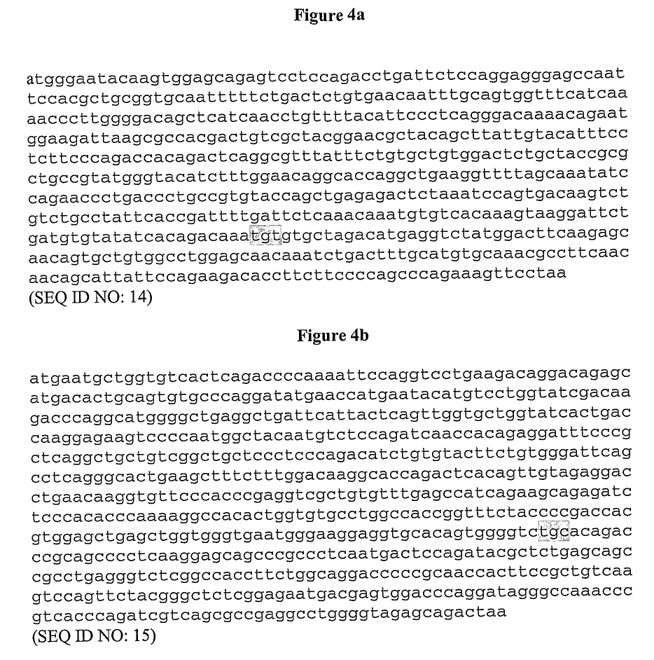

FIGS. 4a and 4b show the DNA sequence of α and β chain of the high affinity c13c1 variant of the Telomerase TCR mutated to include additional cysteine residues to form a non-native disulfide bond, the introduced cysteine is indicated by shading;

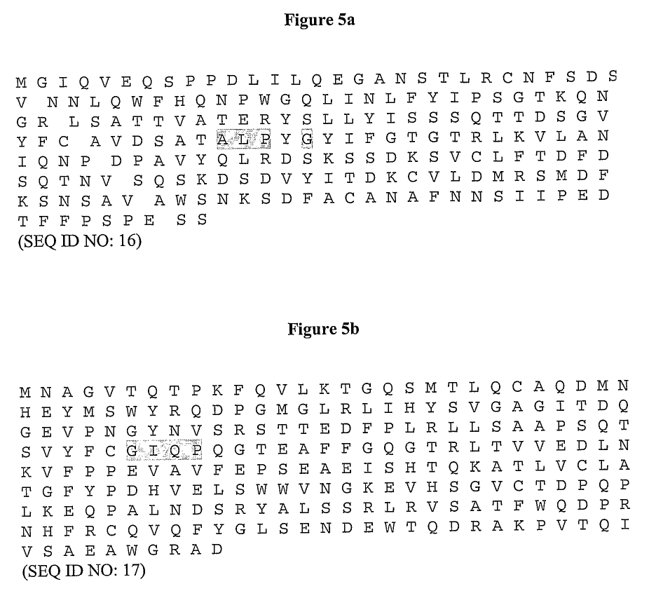

FIGS. 5a and 5b show respectively the c13c1 high affinity variant of the Telomerase TCR α and β chain extracellular amino acid sequences produced from the DNA sequences of FIGS. 4a and 4b the affinity increasing mutations are indicated by shading;

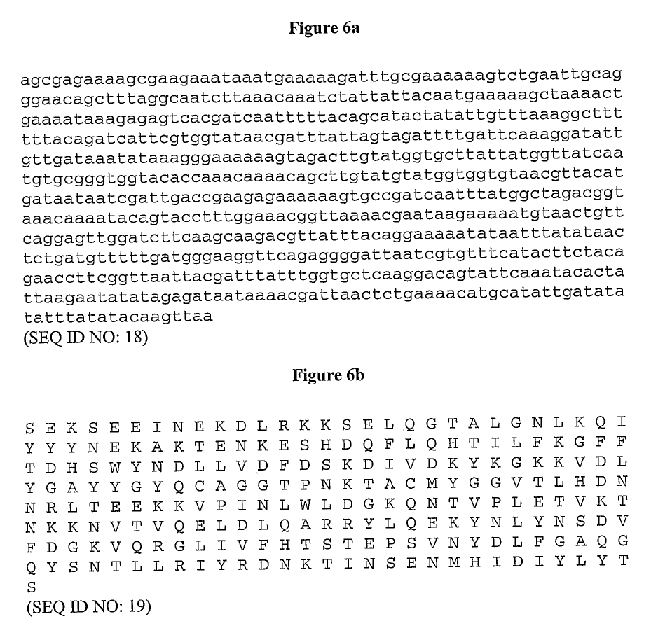

FIG. 6a—DNA sequence of wild-type SEA-E

FIG. 6b—Amino acid sequence of wild-type SEA-E.

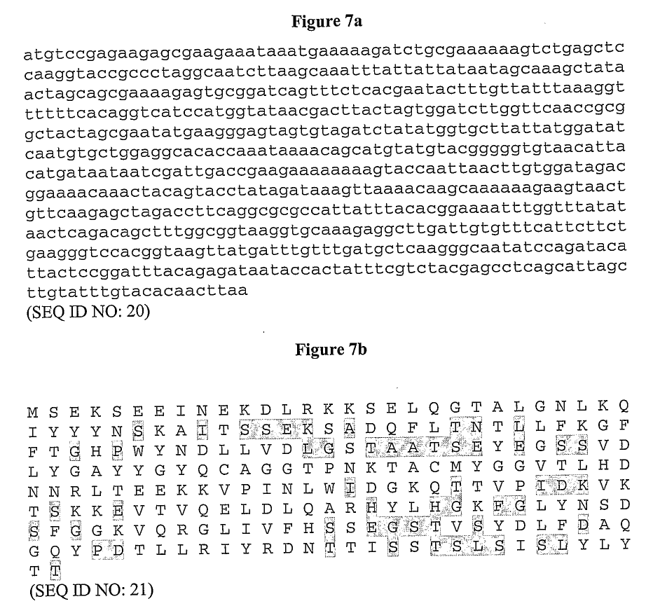

FIG. 7a—DNA sequence of the mutant superantigen SEA-E120.

FIG. 7b—Amino acid sequence of the mutant superantigen SEA-E120, the mutated amino acids are indicated by shading

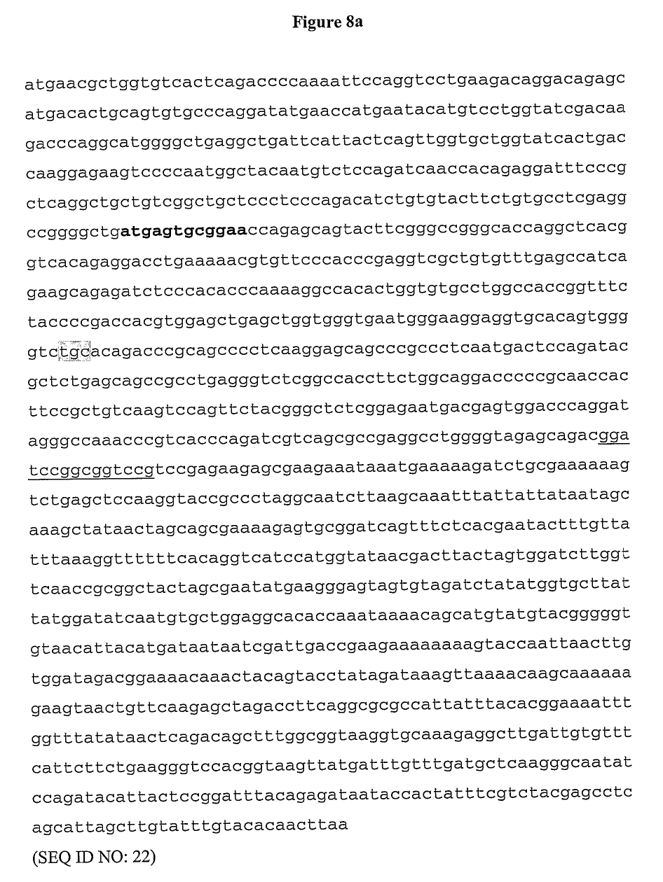

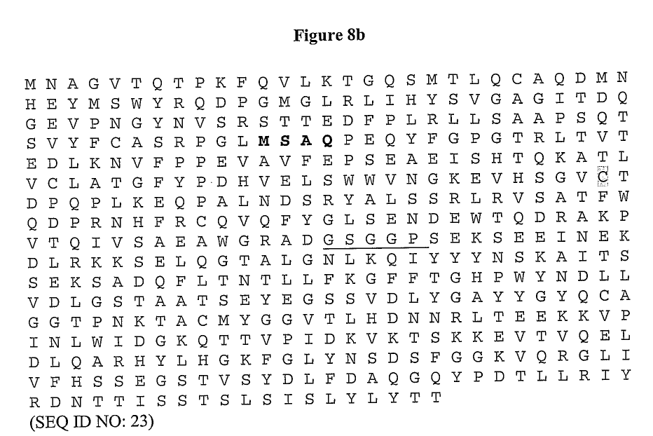

FIG. 8a—DNA sequence of the high affinity c134 variant of the A6 TCR β chain extracellular amino acid sequences containing a non-native cysteine involved in the formation of a novel interchain bond linked to the SEA E120 superantigen via a Gly-Ser-Gly-Gly-Pro linker (L2-linker). (SEQ ID NO: 3) The introduced cysteine codon is indicated by shading. The DNA sequence encoding the Gly-Ser-Gly-Gly-Pro linker is underlined.

FIG. 8b—Amino acid sequence of the high affinity c134 variant of the A6 TCR β chain extracellular amino acid sequences containing a non-native cysteine codon involved in the formation of a novel interchain bond linked to the SEA E120 superantigen via a Gly-Ser-Gly-Gly-Pro linker (L2-linker). (SEQ ID NO: 3) The introduced cysteine is indicated by shading. The Gly-Ser-Gly-Gly-Pro linker is underlined.

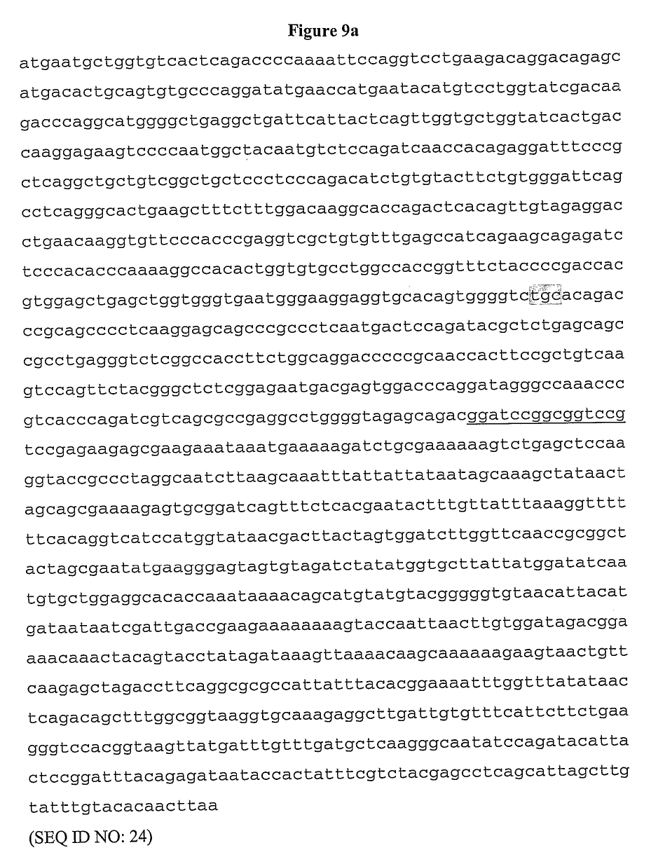

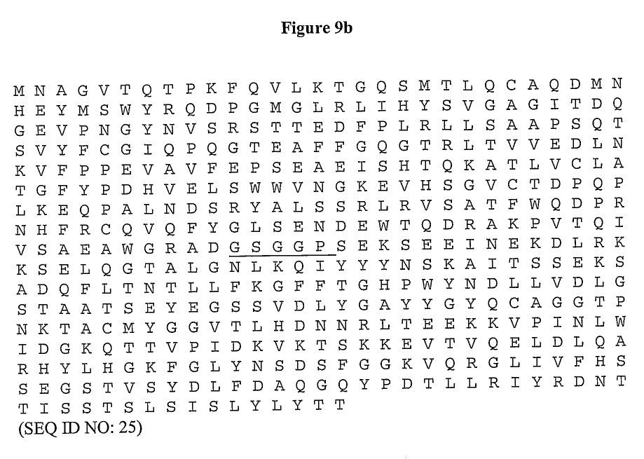

FIG. 9a—DNA sequence of the high affinity c1 variant of a Telomerase TCR β chain extracellular amino acid sequences containing a non-native cysteine involved in the formation of a novel interchain bond linked to the SEA E120 superantigen via a Gly-Ser-Gly-Gly-Pro linker (L2-linker). (SEQ ID NO: 3) The introduced cysteine codon is indicated by shading. The DNA sequence encoding the Gly-Ser-Gly-Gly-Pro linker is underlined.

FIG. 9b—Amino acid sequence of the high affinity cl variant of a Telomerase TCR β chain extracellular amino acid sequences containing a non-native cysteine codon involved in the formation of a novel interchain bond linked to the SEA E120 superantigen via a Gly-Ser-Gly-Gly-Pro linker (L2-linker). (SEQ ID NO: 3) The introduced cysteine is indicated by shading. The Gly-Ser-Gly-Gly-Pro linker is underlined.

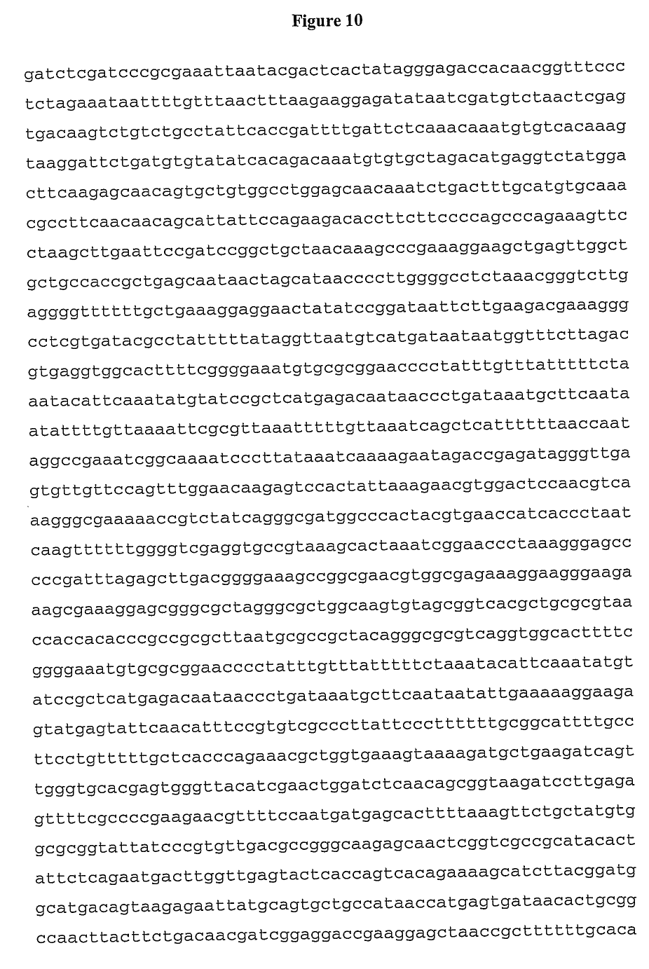

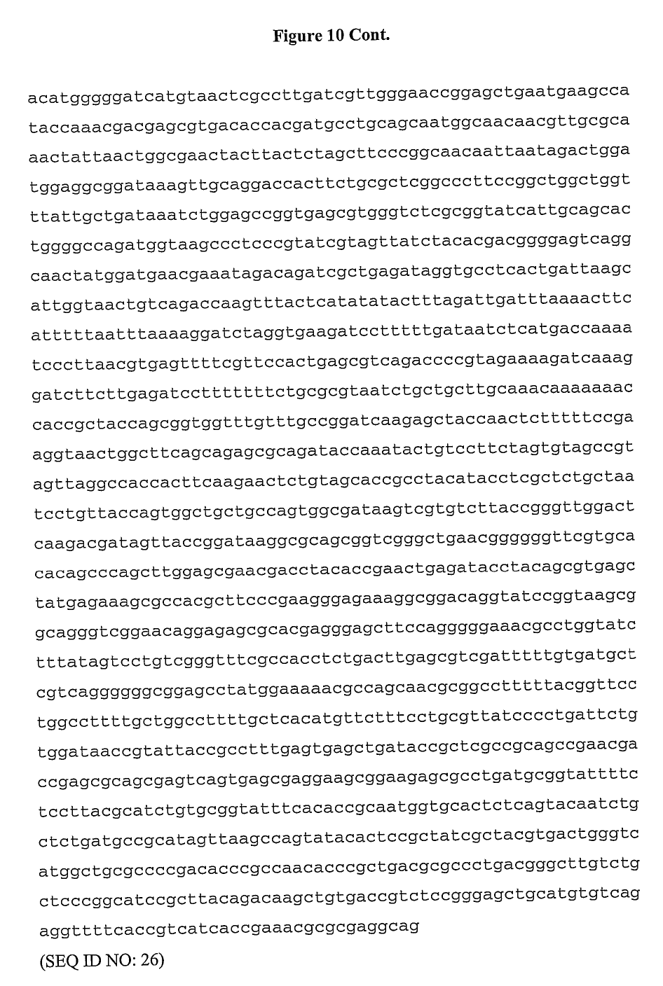

FIG. 10 details the DNA sequence of the pEX954 plasmid

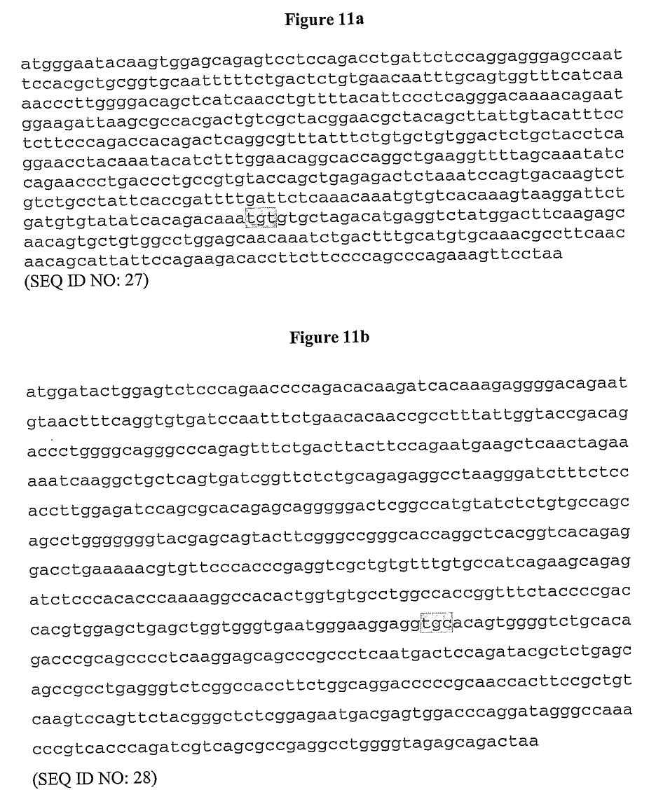

FIGS. 11a and 11b show respectively the DNA sequence encoding the α and β chains of the soluble disulfide-linked TCR comprising a Vβ7.9 domain as used in the TCR-superantigen fusion protein-binding assays. The introduced cysteine codons are indicated by shading.

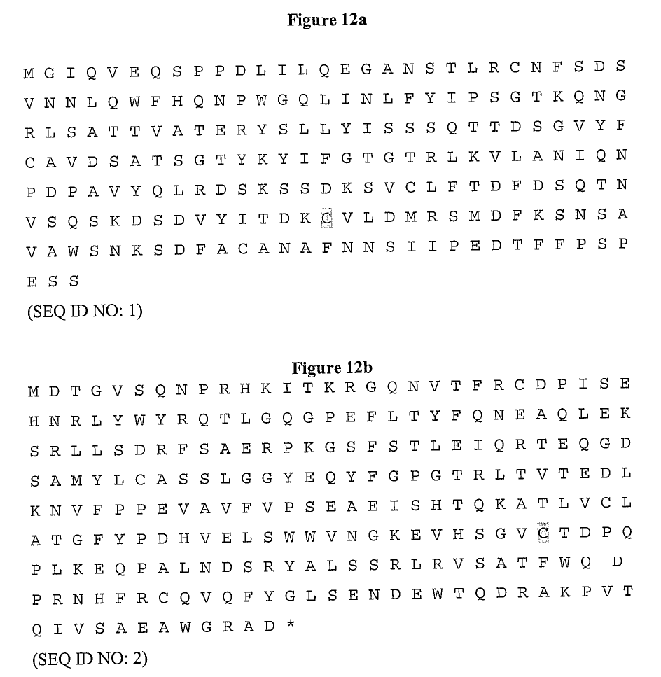

FIGS. 12a and 12b show respectively the amino acid sequence of α and β chain of the soluble disulfide-linked TCR comprising a Vβ7.9 domain as used in the TCR-superantigen fusion protein binding assays and as encoded by the DNA sequences of FIGS. 11a and 11b.The introduced cysteines are indicated by shading.

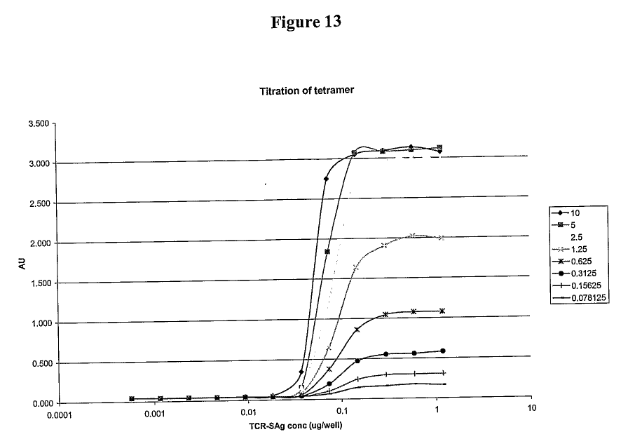

FIG. 13 details the result of titrating TCR tetramer concentration on an ELISA.

FIG. 14 details the binding observed in an ELISA assay of various TCR-superantigen fusion proteins.

FIG. 15 details the EC50 values obtained from the ELISA assay results shown in FIG. 14.

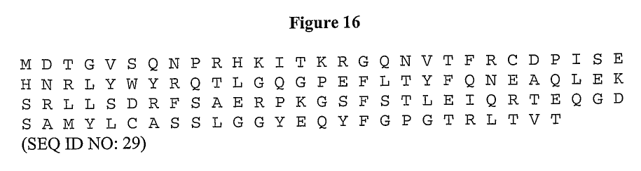

FIG. 16 details the amino acid sequence of a TCR β chain variable region comprising a Vβ7.9 domain.

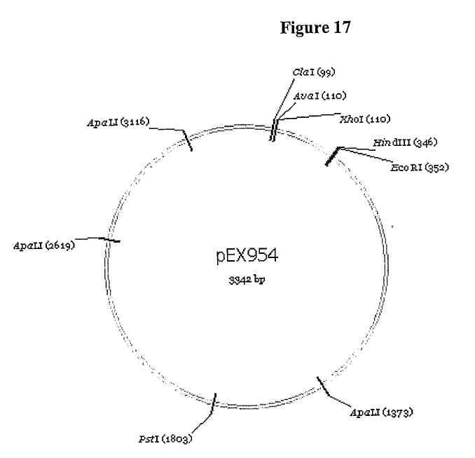

FIG. 17 provides the plasmid map of the pEX954 vector.

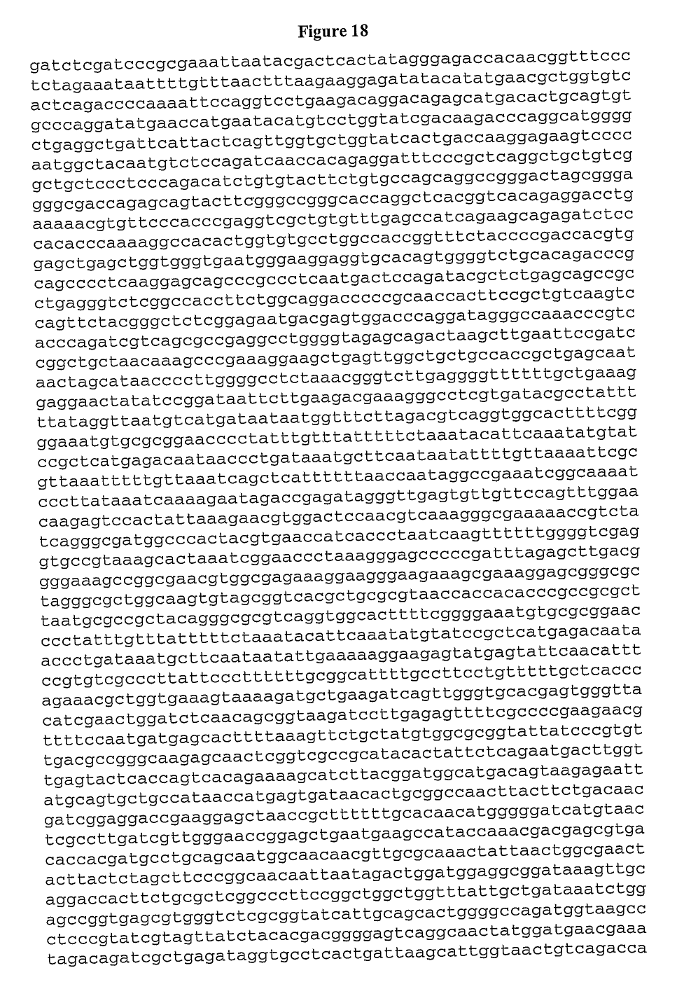

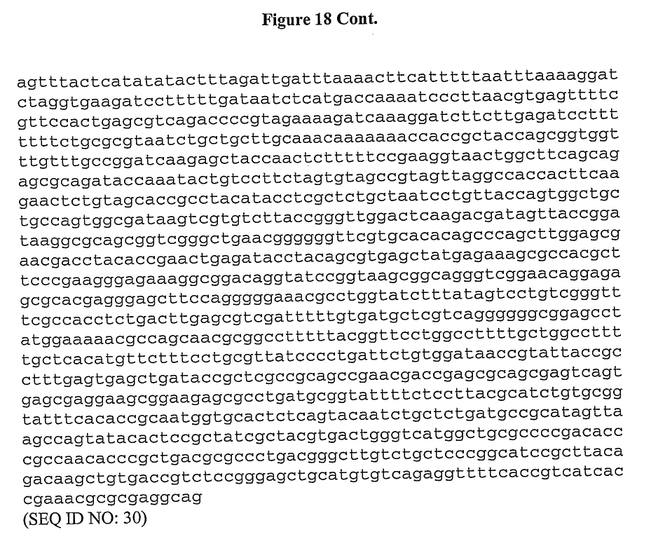

FIG. 18 details the DNA sequence of the pEX821 vector.

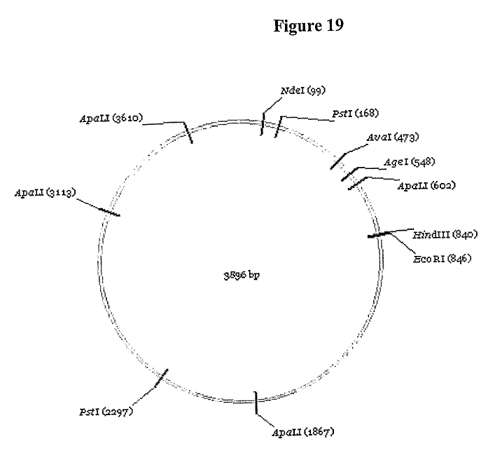

FIG. 19 provides the plasmid map of the pEX821 vector.

### EXAMPLE I

### Production of DNA Encoding a Soluble High Affinity A6 TCR-Superantigen Fusion Protein

Synthetic genes comprising the DNA sequence encoding the soluble high affinity c134A6 TCR β chain detailed in FIG. 3a linked via a DNA sequence encoding a peptide linker to the 5′ end of DNA encoding either the wild-type SEA or mutated SEA E120 superantigens detailed in FIGS. 6a and 7a respectively were synthesised.

There are a number of companies that provide a suitable DNA service, such as Geneart (Germany)

FIG. 8a details the DNA sequence of the high affinity c134 variant of the A6 TCR β chain extracellular amino acid sequences containing a non-native cysteine involved in the formation of a novel interchain bond linked to the SEA E120 superantigen via a Gly-Ser-Gly-Gly-Pro (L2-linker) (SEQ ID NO: 3) The introduced cysteine is indicated by shading. The DNA sequence encoding the Gly-Ser-Gly-Gly-Pro linker is underlined.

FIG. 8b details the amino acid sequence of the high affinity c134 variant of the A6 TCR β chain extracellular amino acid sequences containing a non-native cysteine codon involved in the formation of a novel interchain bond linked to the SEA E120 superantigen via a Gly-Ser-Gly-Gly-Pro (L2 linker) (SEQ ID NO: 3) The introduced cysteine is indicated by shading. The Gly-Ser-Gly-Gly-Pro linker is underlined.

The following are examples linker sequences which may be used for this purpose

ggcggtccg which encodes a Gly-Gly-Pro linker (L1).

ggatccggcggtccg (SEQ ID NO: 4)—which encodes a Gly-Ser-Gly-Gly-Pro (SEQ ID NO: 3) linker (L2) including a BamH1 restriction enzyme site.

ggatccggtgggggcggaagtggaggcagcggtggatccggcggtccg (SEQ ID NO: 5)—which encodes a Gly-Ser-Gly-Gly-Gly-Gly-Ser-Gly-Gly-Ser-Gly-Gly-Ser-Gly-Gly-Pro (SEQ ID NO: 6) linker (L3) including two Bam-H1 restriction enzyme sites.

cccggg—which encodes a Pro-Gly linker (L4) including a Xma1 restriction enzyme site

One of the above synthetic genes encoding the TCR β chain-linker-superantigen fusion protein was then sub-cloned into the pEX821 plasmid. FIG. 18 details the DNA sequence of the pEX821 plasmid and FIG. 19 provides the plasmid map for this vector.

A synthetic gene encoding the α chain of the soluble A6 TCR containing a non-native cysteine codon was then independently sub-cloned into the pEX954 plasmid. Figure 1a details the DNA sequence of this soluble A6 TCR α chain. FIG. 10 details the DNA sequence of the pEX954 plasmid and FIG. 17 provides the plasmid map for this vector.

### EXAMPLE 2

### Production of DNA Encoding a Soluble High Affinity Telomerase TCR-Superantigen Fusion protein

Synthetic genes comprising the DNA sequence encoding the soluble high affinity cl Telomerase TCR β chain detailed in FIG. 4b linked via a DNA sequence encoding a peptide linker to the 5′ end of DNA encoding either the wild-type SEA or mutated SEA E120 superantigens detailed in FIGS. 6a and 7a respectively were synthesised.

There are a number of companies that provide a suitable DNA service, such as Geneart. (Germany)

FIG. 9a—DNA sequence of the high affinity cl variant of a Telomerase TCR β chain extracellular amino acid sequences containing a non-native cysteine involved in the formation of a novel interchain bond linked to the SEA E120 superantigen via a Gly-Ser-Gly-Gly-Pro (L2-linker). (SEQ ID NO: 3) The introduced cysteine is indicated by shading. The DNA sequence encoding the Gly-Ser-Gly-Gly-Pro linker is underlined.

FIG. 9b—Amino acid sequence of the c1 high affinity variant of a Telomerase TCR β chain extracellular amino acid sequences containing a non-native cysteine codon involved in the formation of a novel interchain bond linked to the SEA E120 superantigen via a Gly-Ser-Gly-Gly-Pro (L2) linker. (SEQ ID NO: 3) The introduced cysteine is indicated by shading. The Gly-Ser-Gly-Gly-Pro linker is underlined.

As previously stated a variety of peptide linkers may be suitable to link the TCR β chains to the superantigens. The following are examples linker sequences which may be used for this purpose

The following are examples linker sequences which may be used for this purpose

ggcggtccg which encodes a Gly-Gly-Pro linker (L1).

ggatccggcggtccg (SEQ ID NO: 4)—which encodes a Gly-Ser-Gly-Gly-Pro (SEQ ID NO: 3) linker (L2) including a BamH1 restriction enzyme site. ggatccggtgggggcggaagtggaggcagcggtggatccggcggtccg (SEQ ID NO: 5)—which encodes a Gly-Ser-Gly-Gly-Gly-Gly-Ser-Gly-Gly-Ser-Gly-Gly-Ser-Gly-Gly-Pro (SEQ ID NO: 6) linker (L3) including two BamH1 restriction enzyme sites.

cccggg—which encodes a Pro-Gly linker (L4) including a Xma1 restriction enzyme site

One of the above synthetic genes encoding the TCR β chain-linker-superantigen fusion protein was then sub-cloned into the pEX821 plasmid. FIG. 18 details the DNA sequence of this plasmid and FIG. 19 provides the corresponding plasmid map.

A synthetic gene encoding the α chain of the soluble Telomerase TCR containing a non-native cysteine codon was then independently sub-cloned into the pEX954 plasmid. FIG. 4a details the DNA sequence of this soluble Telomerase TCR α chain.

As will be obvious to those skilled in the art the methods described in Examples 1 and 2 may be used to produce soluble TCR-superantigen fusion proteins of the invention from any TCR for which the DNA sequence is known.

### EXAMPLE 3

### Expression, Refolding and Purification of Soluble TCR-superantigen Fusion Proteins

The pEX954 and pEX821 expression plasmids containing the mutated TCR α-chain and TCR β-chain—superantigen fusion proteins respectively were transformed separately into E. coli strain BL21pLysS, and single ampicillin-resistant colonies were grown at 37° C. in TYP (ampicillin 100 μg/ml) medium to OD₆₀₀ of 0.4 before inducing protein expression with 0.5 mM IPTG. Cells were harvested three hours post-induction by centrifugation for 30 minutes at 4000 rpm in a Beckman J-6B. Cell pellets were re-suspended in a buffer containing 50 mM Tris-HCI, 25% (w/v) sucrose, 1 mM NaEDTA, 0.1% (w/v) NaAzide, 10 mM DTT, pH 8.0. After an overnight freeze-thaw step, re-suspended cells were sonicated in 1 minute bursts for a total of around 10 minutes in a Milsonix XL2020 sonicator using a standard 12 mm diameter probe. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenised in a Triton buffer (50 mM Tris-HCI, 0.5% Triton-X100, 200 mM NaCI, 10 mM NaEDTA, 0.1% (w/v) NaAzide, 2 mM DTT, pH 8.0) before being pelleted by centrifugation for 15 minutes at 13000 rpm in a Beckman J2-21. Detergent and salt was then removed by a similar wash in the following buffer: 50 mM Tris-HCl, 1 mM NaEDTA, 0.1% (w/v) NaAzide, 2 mM DTT, pH 8.0. Finally, the inclusion bodies were divided into 30 mg aliquots and frozen at −70° C. Inclusion body protein yield was quantitated by solubilising with 6 M guanidine-HCl and measurement with a Bradford dye-binding assay (PerBio).

Denaturation of soluble polypeptides; 30 mg of the solubilised TCR β-chain-superantigen inclusion body and 60 mg of the solubilised TCR α-chain inclusion body was thawed from frozen stocks. The inclusion bodies were diluted to a final concentration of 5 mg/ml in 6 M guanidine solution, and DTT (2 M stock) was added to a final concentration of 10 mM. The mixture was incubated at 37° C. for 30 min. Refolding of soluble TCR-superantigen fusion proteins: 1 L refolding buffer was stirred vigorously at 5° C.±3° C. The redox couple (2-mercaptoethylamine and cystamine (to final concentrations of 6.6 mM and 3.7 mM, respectively) were added approximately 5 minutes before addition of the denatured TCR/TCR-superantigen polypeptides. The protein was then allowed to refold for approximately 5 hours±15 minutes with stirring at 5° C.±3° C.

Dialysis of refolded soluble TCR-superantigen fusion proteins: The refolded TCR-superantigen fusion proteins was dialysed in Spectrapor 1 membrane (Spectrum;

Product No. 132670) against 10 L 10 mM Tris pH 8.1 at 5° C.±3° C. for 18-20 hours. After this time, the dialysis buffer was changed to fresh 10 mM Tris pH 8.1 (10 L) and dialysis was continued at 5° C.±3° C. for another 20-22 hours.

### EXAMPLE 4

### Preparation of Biotinylated Disulfide-Linked Soluble TCRs Containing a Vβ7.9 Variable Domain, and Tetramers Thereof

Biotinylated soluble TCR monomer production

A recognition tag DNA sequence (GGA TCC GGT GGT GGT CTG AAC GAT ATT TTT GAA GCT CAG AAA ATC GAA TGG CAT) (SEQ ID NO: 7) can be inserted into the 3′ end of any given soluble TCR α or TCR β chain DNA sequence immediately up-stream of the existing stop (taa) codon. This will allow the production of a soluble TCR containing a biotin recognition tag which can be expressed and refolded using the methods described in Examples 1-3.

FIGS. 11a and 11b respectively detail the DNA sequence of α and β chain of a soluble TCR, FIGS. 12a and 12b show respectively the amino acid sequence ofα and β chain of this soluble TCR as encoded by the DNA sequences of FIGS. 11a and 11b. The above biotin recognition tag DNA sequence was inserted into the 3′ end of the TCR β chain DNA sequence detailed in FIG. 11b immediately before the existing stop (taa) codon. The soluble TCR containing a biotin recognition tag was biotinylated as follows: 2.5 ml of purified soluble TCR solution (˜0.2 mg/ml) was buffer exchanged into biotinylation reaction buffer (50 mM Tris pH 8.0, 10 mM MgCl₂) using a PD-10 column (Pharmacia). The eluate (3.5 ml) was concentrated to 1 ml using a centricon concentrator (Amicon) with a 10 kDa molecular weight cut-off. This was made up to 10 mM with ATP added from stock (0.1 g/ml adjusted to pH 7.0). A volume of a cocktail of protease inhibitors was then added (protease inhibitor cocktail Set 1, Calbiochem Biochemicals ), sufficient to give a final protease cocktail concentration of 1/100^{th} of the stock solution as supplied, followed by 1 mM biotin (added from 0.2 M stock) and 20 μg/ml enzyme (from 0.5 mg/ml stock). The mixture was then incubated overnight at room temperature. Excess biotin was removed from the solution by size exclusion chromatography on a S75 HR column. The level of biotinylation present on the soluble TCRs was determined via a size exclusion HPLC-based method as follows. A 50 ul aliquot of the biotinylated soluble TCR (2 mg/ml) was incubated with 50 ul of streptavidin coated agarose beads (Sigma) for 1 hour. The beads were then spun down, and 50 μl of the unbound sample was run on a TSK 2000 SW column (Tosoohaas) using a 0.5 ml/min flow-rate (200 mM Phosphate Buffer pH 7.0) over 30 minutes. The presence of the biotinylated soluble TCR was detected by a UV spectrometer at both 214 nm and 280 nm. The biotinylated soluble TCR was run against a non-biotinylated soluble TCR control. The percentage of biotinylation was calculated by subtracting the peak-area of the biotinylated protein from that of the non-biotinylated protein. Aliquots of biotinylated TCR monomers were stored frozen at −20° C.

TCR Tetramer Preparation

Tetramerisation of the biotinylated soluble TCR was achieved using streptavidin. The concentration of biotinylated soluble TCR was measured using a Coomassie protein assay (Pierce), and the quantities of the soluble TCR and streptavidin required to ensure a 1:4 molar ratio of soluble TCR:streptavidin were calculated. The biotinylated soluble TCR solution in phosphate buffered saline (PBS) was added slowly to a

1 mg/ml streptavidin solution over ice with gentle agitation. 100.5 μl of PBS was then added to this solution to provide a final TCR tetramer concentration of 1 mg/ml.

### EXAMPLE 5

### BIAcore surface plasmon resonance characterisation of the binding of TCR-superantigen Fusion Proteins to Soluble Disulfide-Linked TCRs

A surface plasmon resonance biosensor (BIAcore 300™) was used to analyse the binding of TCR-superantigen fusion proteins to soluble disulfide-linked TCRs.

The soluble biotinylated TCR monomer prepared as described in Example 4 was immobilised to fresh CM5 chips which had been primed with BIA-HBS-EP buffer and coated with ˜5000RU of Streptavidin. TCR coupling densities were as follows:

| | | | After |
|---|---|---|---|
| FC | Ligand | Initial | regeneration |
| 1 | Blank | −8 | −44 |
| 3 | VB7.9 | 512 | 465 |
| | TCR | | |

The BIAcore 3000 was run at 20 ul/min, using quickinjects of 20 ul of each TCR-superantigen fusion protein. Regeneration was with 5 ul of 50 mM NaOH, then 5 ul of 10 mM NaOH, followed by a 5 ul quickinjection of HBS to thoroughly flush out the needle.

TCR-Superantigen Fusion Proteins Assessed:

| | | TCR- | | | |
|---|---|---|---|---|---|
| | | SAg | Extinction | | Top Conc. |
| | OD280 | (mg/ml) | factor | MW | (nM) |
| Tel c13c1-L1- | 1.96 | | 0.72 | 76200 | 18520 |
| SEA/E-120 (Fusion | | | | | |
| b) | | | | | |
| Tel c13c1-L2- | | 1.2 | | 76200 | 15748 |
| SEA/E-120 (Fusion | | | | | |
| c) | | | | | |
| Tel c13c1-L3- | | 0.8 | | 76200 | 10499 |
| SEA/E-120 (Fusion | | | | | |
| d) | | | | | |
| A6 Tax cwtc134- | 5.892 | | 0.72 | 76500 | 55454 |
| L2-SEA/E-120 | | | | | |
| (Fusion a, Batch 1) | | | | | |

The response curves were aligned on the X-axis, and the response from the blank FC1 chip was subtracted. All readings for the steady state affinity measurements were taken 50 seconds into the one-minute quickinjections.

Results

| Fusion | | | | TAX cwtc134- |
|---|---|---|---|---|
| protein | TEL c13c1-SEA/E-120 | | | SEA/E-120 |
| Linker | 1 | 2 | X | 2 |
| KD (nM) | 1050 | 1047 | 2419 | 914 |
| Std. Error | 65.28 | 60.17 | 70.84 | 58.96 |
| Regeneration | NaOH | NaOH | NaOH | NaOH |

### EXAMPLE 6

### In-vitro ELISA assay of high affinity A6 TCR-SEA E-120

A 23 μg/ml solution of the high affinity cwtc134 A6 TCR-L2-SEA/E-120 fusion protein in PBS was prepared. FIGS. 2a and 8b detail the amino acid sequences of the wt A6 TCR α chain and the c134 TCR β chain-L2-SEA-E120 fusion polypeptide respectively.

50 μl/well (1.1 8 μg/well) of this solution was added into columns 1 and 2 of a Nunc Maxisorp plate (Plate 1). 50 μl of PBS was then added to columns 2-12 of this plate, and 50 ul of the resulting solution was transferred from column 2, mixed and serially diluted across the plate to column 11. Column 12 was left blank. This plate was prepared in order to ascertain the most appropriate quantity of TCR Vβ7.9 tetramer, prepared as described in Example 4, to use for assays to determine the overall quality/activity of TCR-superantigen batches. This was assessed by adding a range of TCR tetramer quantities to the well in Plate 1.

A second Nunc Maxisorp plate (Plate 2) was then prepared as follows. 50 μl of PBS was added to columns 2-12. The following samples were then added to columns 1 and 2, and subsequently diluted across the plate:

| | Starting Conc. | TCR-Sag |
|---|---|---|
| Fusion Protein | μg/well | nM |
| A6 Tax cwtc134-L2-SEA/E- | 1.180 | 308 |
| 120 | | |
| (Fusion a, Batch 1) | | |
| Blank | 0.000 | 0 |
| Tel c13c1-L1-SEA/E-120 | 5.67 | 1481 |
| (Fusion b) | | |
| Tel c13c1-L2-SEA/E-120 | 2.4 | 627 |
| (Fusion c) | | |
| Tel c13c1-L3-SEA/E-120 | 3.33 | 871 |
| (Fusion d) | | |
| 3xFT A6 Tax cwtc134-L2- | 1.18 | 308 |
| SEA/E-120 (Fusion e- | | |
| Freeze/thaw treated Fusion a) | | |
| 50° C. A6 Tax cwtc134-L2- | 1.18 | 308 |
| SEA/E-120 | | |
| (Fusion f - heat treated | | |
| Fusion a) | | |
| A6 Tax cwtc134-L2-SEA/E- | 4.24 | 1108 |
| 120 (Fusion g - 2^{nd} batch of | | |
| Fusion a) | | |

Plate 2 was prepared in order to test the ability of the assay to quantify the overall 15 assess the overall quality of the superantigen part of a range of different TCR-Superantigen fusions as well as freeze/thaw and heat treated samples of Fusion a.

These plates were incubated overnight at 4° C.

ELISA Assay

Both plates were washed 3× (with PBST) and blocked (with 200 μl/well PBS 2% BSA) for 2 hours at 4° C. A titration of TCR tetramer was made in a round-bottomed 96-well plate. 1.1 ug (0.5 ml in PBS 1% BSA) aliquot of Vβ7.9 tetramer was thawed and diluted to 5.5 ml with PBS 1% BSA. This went into rows 1 and 2 (100 μl per well), and 100 μl PBS 1% BSA was added to rows 2 to 8. The tetramer was diluted down the plate. Plate 1 was then washed 3 times (with PBST) and 50 ul/well tetramer was added. This plate was then incubated at 4° C. for 1 hour, before washing 6 times (with PBST) and developing with TMB peroxidase substrate system (KPL, product number 50-76-00). The 100 μl/well of TMB mix was added and incubated on the plate shaker platform for 20 minutes before the reaction was stopped with 100 μl/well IM H₂SO₄. The plate was then read at 450 nm using a Wallac Victor II plate reader.

FIG. 13 details the ELISA responses observed across the range of TCR tetramer concentrations used. This titration data revealed that a total of 1.25 ng/well TCR tetramer gave the optimal response in terms of being able to determine EC50 values. (Concentration of TCR tetramer required to reach half maximal binding) The EC50 value of a given TCR-Superantigen will be used as a measure of the overall quality/activity of the superantigen part of superantigen-containing compounds.

1.25 ng/well of TCR tetramer was then added to each well of Plate 2.

The following table details the ELISA-determined EC50 values for each of the TCR-superantigen fusion polypeptides assessed:

| TCR- | | | | | | | |
|---|---|---|---|---|---|---|---|
| Sag | Fusion a | Fusion B | Fusion C | Fusion D | Fusion e | Fusion f | Fusion G |
| EC50 | 25.6 | 25.3 | 17.3 | 71.5 | 42.59 | 38.68 | 21.7 |
| (nM) | | | | | | | |

FIG. 14 details the ELISA responses observed for each of the TCR-superantigen fusions tested.

FIG. 15 details the ELISA-determined EC50 values determined from the results shown in FIG. 14 for each of the TCR-superantigen fusion proteins tested. The EC50 value for each fusion protein includes the respective 95% confidence limits.

These data demonstrate that this ELISA assay is capable of providing an EC50 value for each of the TCR-superantigen samples assessed.

The EC50 results obtained for the following of the TCR-superantigen fusion samples assessed were compared to results obtained using the Biacore-based method described in Example 5:

| TCR-superantigen | ELISA EC50 | Biacore K_{D} |
|---|---|---|
| Fusion | (nM TCR-SAg) | (nM) |
| Fusion a, batch 1 | 25.6 | 914 |
| Fusion b | 25.3 | 1050 |
| Fusion c | 17.3 | 1047 |
| Fusion d | 71.5 | 2419 |

The above results demonstrate that both the ELISA and Biacore-based assays can provide data which can be used to assess the superantigen part of a superantigen containing composition. Also, both methods are in broad agreement in terms of the relative overall binding response generated by the superantigen part of the superantigen-containing compositions assessed.

1. A heterodimeric TCR (dTCR) or single-chain TCR (scTCR) comprising SEQ ID NO: 29 and which binds to SEA-E120 having SEQ ID NO: 21.

2. A dTCR or scTCR comprising the TCR β chain sequence of SEQ ID NO: 2 and which binds to SEA-E120 having SEQ ID NO: 21.

3. A dTCR as claimed in claim 1 comprising the TCR α chain amino acid sequence of SEQ ID NO: 1 and the TCR β chain sequence of SEQ ID NO: 2.

4. A superantigen assay comprising incubating a standard amount of a superantigen-containing test sample with a standard amount of a soluble TCR which binds the superantigen, separating unbound TCR from the resultant superantigen/TCR-containing sample, quantifying the TCR bound in that sample, and comparing that result with a reference result characterising a control superantigen-containing sample.

5. An assay as claimed in claim 4 wherein the superantigen is SEA-E120 having SEQ ID NO: 21.

6. An assay as claimed in claim 4 wherein the assay is performed on a series of aliquots of the superantigen-containing test sample, each aliquot containing a different amount of the said sample, and the bound TCR result for comparison with the reference result is estimated as a function of the individual quantifications of the bound TCR in each aliquot.

7. An assay as claimed in claim 4 wherein the reference result is the result of the same assay performed on a control superantigen-containing sample.

8. An assay as claimed in claim 4 wherein a multimeric TCR is used.

9. An assay as claimed in claim 4 wherein a tetrameric TCR is used.

10. An assay as claimed in claim 4 wherein the said quantification is by an Interfacial Optical Assay.

11. An assay as claimed in claim 10 wherein the said quantification is by Surface Plasmon Resonance (SPR).

12. An assay as claimed in claim 4 wherein the said quantification is by an Enzyme-Linked Immunosorbent Assay (ELISA).

13. An assay as claimed in claim 4 wherein the TCR comprises
a first polypeptide wherein a sequence corresponding to a TCR α chain variable region sequence fused to the N terminus of a sequence corresponding to a TCR α chain constant region extracellular sequence, and
a second polypeptide wherein a sequence corresponding to a TCR β chain variable region sequence fused to the N terminus a sequence corresponding to a TCR β chain constant region extracellular sequence,
the first and second polypeptides being linked by a disulfide bond between cysteine residues substituted for Thr 48 of exon 1 of TRAC*01 and Ser 57 of exon 1 of TRBC1*01 or TRBC2*01 or the non-human equivalent thereof.

14. An assay as claimed in claim 4 wherein the TCR comprises the TCR α chain amino acid sequence of SEQ ID NO: 1 and the TCR β chain sequence of SEQ ID NO: 2.


SEQUENCE LISTING (30 sequences)

SEQ ID NO: 1 - protein, 205 aa, Artificial sequence
  Amino acid sequence of preferred soluble TCR alpha chain
MGIQVEQSPPDLILQEGANSTLRCNFSDSVNNLQWFHQNPWGQLINLFYIPSGTKQNGRLSATTVATERYSLLYISSSQTTDSGVYFCAVDSATSGTYKYIFGTGTRLKVLAIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 2 - protein, 243 aa, Artificial sequence
  Amino acid sequence of preferred soluble TCR beta chain
MDTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLGGYEQYFGPGTRLTVTEDLKNVFPPEVAVFVPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 3 - protein, 5 aa, Artificial sequence
  L2 Linker amino acid sequence
GSGGP

SEQ ID NO: 4 - DNA, 15 bp, Artificial sequence
  DNA encoding L2 Linker
ggatccggcggtccg

SEQ ID NO: 5 - DNA, 48 bp, Artificial sequence
  DNA encoding L3 linker
ggatccggtgggggcggaagtggaggcagcggtggatccggcggtccg

SEQ ID NO: 6 - protein, 16 aa, Artificial sequence
  L3 Linker amino acid sequence
GSGGGGSGGSGGSGGP

SEQ ID NO: 7 - DNA, 54 bp, Artificial sequence
  DNA encoding a biotin recognition tag
ggatccggtggtggtctgaacgatatttttgaagctcagaaaatcgaatggcat

SEQ ID NO: 8 - DNA, 621 bp, Artificial sequence
  DNA encoding soluble DiS-linked A6 TCR alpha chain
atgcagaaggaagtggagcagaactctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgttacaactgacagctgggggaaattgcagtttggagcagggacccaggttgtggtcaccccagatatccagaacccggatcctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 9 - DNA, 741 bp, Artificial sequence
  DNA encoding soluble DiS-linked A6 TCR beta chain
atgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaggccgggactagcgggagggcgaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 10 - protein, 206 aa, Artificial sequence
  Amino acid sequence of soluble DiS-linked A6 TCR alpha chain
MQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 11 - protein, 246 aa, Artificial sequence
  Amino acid sequence of soluble DiS-linked A6 TCR beta chain
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLAGGRPEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 12 - DNA, 741 bp, Artificial sequence
  DNA sequence of soluble DiS-linked high affinity c134 A6 TCR beta chain
atgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgcctcgaggccggggctgatgagtgcggaaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 13 - protein, 246 aa, Artificial sequence
  Amino acid sequence of soluble DiS-linked high affinity c134 A6 TCR beta chain
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLMSAQPEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 14 - DNA, 621 bp, Artificial sequence
  DNA sequence of soluble DiS-linked high affinity c13 Telomerase TCR alpha chain
atgggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggactctgctaccgcgctgccgtatgggtacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 15 - DNA, 729 bp, Artificial sequence
  DNA sequence of soluble DiS-linked high affinity c1 Telomerase TCR beta chain
atgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgggattcagcctcagggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 16 - protein, 206 aa, Artificial sequence
  Amino acid sequence of DiS linked high affinity c13 Telomerase TCR alpha chain
MGIQVEQSPPDLILQEGANSTLRCNFSDSVNNLQWFHQNPWGQLINLFYIPSGTKQNGRLSATTVATERYSLLYISSSQTTDSGVYFCAVDSATALPYGYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 17 - protein, 242 aa, Artificial sequence
  Amino acid sequence of DiS linked high affinity c13 Telomerase TCR beta Chain
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCGIQPQGTEAFFGQGTRLTVVEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 18 - DNA, 702 bp, Staphylococcus aureus
agcgagaaaagcgaagaaataaatgaaaaagatttgcgaaaaaagtctgaattgcagggaacagctttaggcaatcttaaacaaatctattattacaatgaaaaagctaaaactgaaaataaagagagtcacgatcaatttttacagcatactatattgtttaaaggcttttttacagatcattcgtggtataacgatttattagtagattttgattcaaaggatattgttgataaatataaagggaaaaaagtagacttgtatggtgcttattatggttatcaatgtgcgggtggtacaccaaacaaaacagcttgtatgtatggtggtgtaacgttacatgataataatcgattgaccgaagagaaaaaagtgccgatcaatttatggctagacggtaaacaaaatacagtacctttggaaacggttaaaacgaataagaaaaatgtaactgttcaggagttggatcttcaagcaagacgttatttacaggaaaaatataatttatataactctgatgtttttgatgggaaggttcagaggggattaatcgtgtttcatacttctacagaaccttcggttaattacgatttatttggtgctcaaggacagtattcaaatacactattaagaatatatagagataataaaacgattaactctgaaaacatgcatattgatatatatttatatacaagttaa

SEQ ID NO: 19 - protein, 233 aa, Staphylococcus aureus
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 20 - DNA, 705 bp, Artificial sequence
  DNA encoding mutated SEA-E120 Superantigen
atgtccgagaagagcgaagaaataaatgaaaaagatctgcgaaaaaagtctgagctccaaggtaccgccctaggcaatcttaagcaaatttattattataatagcaaagctataactagcagcgaaaagagtgcggatcagtttctcacgaatactttgttatttaaaggttttttcacaggtcatccatggtataacgacttactagtggatcttggttcaaccgcggctactagcgaatatgaagggagtagtgtagatctatatggtgcttattatggatatcaatgtgctggaggcacaccaaataaaacagcatgtatgtacgggggtgtaacattacatgataataatcgattgaccgaagaaaaaaaagtaccaattaacttgtggatagacggaaaacaaactacagtacctatagataaagttaaaacaagcaaaaaagaagtaactgttcaagagctagaccttcaggcgcgccattatttacacggaaaatttggtttatataactcagacagctttggcggtaaggtgcaaagaggcttgattgtgtttcattcttctgaagggtccacggtaagttatgatttgtttgatgctcaagggcaatatccagatacattactccggatttacagagataataccactatttcgtctacgagcctcagcattagcttgtatttgtacacaacttaa

SEQ ID NO: 21 - protein, 234 aa, Artificial sequence
  Mutated SEA-E120 Superantigen
MSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 22 - DNA, 1455 bp, Artificial sequence
  DNA encoding soluble high affinity c134 variant A6 TCR beta chain-L2 Linker-SEA E120 fusion protein
atgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgcctcgaggccggggctgatgagtgcggaaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggatccggcggtccgtccgagaagagcgaagaaataaatgaaaaagatctgcgaaaaaagtctgagctccaaggtaccgccctaggcaatcttaagcaaatttattattataatagcaaagctataactagcagcgaaaagagtgcggatcagtttctcacgaatactttgttatttaaaggttttttcacaggtcatccatggtataacgacttactagtggatcttggttcaaccgcggctactagcgaatatgaagggagtagtgtagatctatatggtgcttattatggatatcaatgtgctggaggcacaccaaataaaacagcatgtatgtacgggggtgtaacattacatgataataatcgattgaccgaagaaaaaaaagtaccaattaacttgtggatagacggaaaacaaactacagtacctatagataaagttaaaacaagcaaaaaagaagtaactgttcaagagctagaccttcaggcgcgccattatttacacggaaaatttggtttatataactcagacagctttggcggtaaggtgcaaagaggcttgattgtgtttcattcttctgaagggtccacggtaagttatgatttgtttgatgctcaagggcaatatccagatacattactccggatttacagagataataccactatttcgtctacgagcctcagcattagcttgtatttgtacacaacttaa

SEQ ID NO: 23 - protein, 484 aa, Artificial sequence
  Amino acid sequence of soluble high affinity c134 variant A6 TCR beta chain-L2 Linker-SEA E120 fusion protein
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLMSAQPEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 24 - DNA, 1443 bp, Artificial sequence
  DNA encoding soluble high affinity c1 Telomerase TCR beta chain-L2 Linker-SEA E120 fusion protein
atgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgggattcagcctcagggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggatccggcggtccgtccgagaagagcgaagaaataaatgaaaaagatctgcgaaaaaagtctgagctccaaggtaccgccctaggcaatcttaagcaaatttattattataatagcaaagctataactagcagcgaaaagagtgcggatcagtttctcacgaatactttgttatttaaaggttttttcacaggtcatccatggtataacgacttactagtggatcttggttcaaccgcggctactagcgaatatgaagggagtagtgtagatctatatggtgcttattatggatatcaatgtgctggaggcacaccaaataaaacagcatgtatgtacgggggtgtaacattacatgataataatcgattgaccgaagaaaaaaaagtaccaattaacttgtggatagacggaaaacaaactacagtacctatagataaagttaaaacaagcaaaaaagaagtaactgttcaagagctagaccttcaggcgcgccattatttacacggaaaatttggtttatataactcagacagctttggcggtaaggtgcaaagaggcttgattgtgtttcattcttctgaagggtccacggtaagttatgatttgtttgatgctcaagggcaatatccagatacattactccggatttacagagataataccactatttcgtctacgagcctcagcattagcttgtatttgtacacaacttaa

SEQ ID NO: 25 - protein, 480 aa, Artificial sequence
  Amino acid sequence of soluble high affinity c1 Telomerase TCR beta chain- L2 Linker-SEA E120 fusion protein
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCGIQPQGTEAFFGQGTRLTVVEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 26 - DNA, 3342 bp, Artificial sequence
  PEX954 vector
gatctcgatcccgcgaaattaatacgactcactatagggagaccacaacggtttccctctagaaataattttgtttaactttaagaaggagatataatcgatgtctaactcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtgaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 27 - DNA, 621 bp, Artificial sequence
  DNA encoding alpha chain of preferred soluble DiS-linked TCR
atgggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggactctgctacctcaggaacctacaaatacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 28 - DNA, 732 bp, Artificial sequence
  DNA encoding beta chain of preferred soluble DiS-linked TCR
atggatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcctgggggggtacgagcagtacttcgggccgggcaccaggctcacggtcacagaggacctgaaaaacgtgttcccacccgaggtcgctgtgtttgtgccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 29 - protein, 113 aa, Artificial sequence
  Amino acid sequence of a TCR beta chain variable region comprising a V 7.9 domain.
MDTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLGGYEQYFGPGTRLTVT

SEQ ID NO: 30 - DNA, 3836 bp, Artificial sequence
  pEX821 vector
gatctcgatcccgcgaaattaatacgactcactatagggagaccacaacggtttccctctagaaataattttgtttaactttaagaaggagatatacatatgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaggccgggactagcgggagggcgaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag